(19) United States
(12) Patent Application Publication
(10) Pub. No.: US 2012/0324736 A1
(43) Pub. Date: Dec. 27, 2012

(54) Hair Removal Device Comprising Erodable Moisturizer Comprising An Anti-Irritation Agent

(76) Inventor: Christopher Raymond Eagleton, Devizes (GB)

(21) Appl. No.: 13/529,867

(22) Filed: Jun. 21, 2012

Related U.S. Application Data
(60) Provisional application No. 61/524,097, filed on Aug. 16, 2011.

(30) Foreign Application Priority Data
Jun. 24, 2011 (EP) 11171369.9

(51) Int. Cl. B26B 21/44; B26B 21/22; A61Q 9/02; A61K 8/02; A61K 8/58

(52) U.S. Cl. 30/41; 424/401; 424/73

(57) ABSTRACT

A razor cartridge is provided comprising a housing having a base which is connectable to a handle, two or more blades, whose tips are aligned in a cutting plane to cut in a cutting direction, and a moisturizing block disposed before and/or after the blades in the cutting direction, the moisturizing block having a skin-contacting surface, wherein: the moisturizing block comprises at least 50% lipophilic materials by weight of the moisturizing block; and from about from about 0.01% to 5% of an anti-irritation agent comprising a pyrithione, a polyvalent metal salt of pyrithione, and a mixture thereof, such as zinc pyrithione.

### CROSS REFERENCE TO RELATED APPLICATION(S)

This application claims priority to U.S. Application No. 61/524,097, filed Aug. 16, 2011 and U.S. application Ser. No. 13/528,932 filed on Jun. 21, 2012, which claims priority to EP Application No. 11171369.9, filed Jun. 24, 2011, the subject of which is hereby incorporated by reference in its entirety.

### BACKGROUND OF THE INVENTION

Hair removal devices incorporating a chemical composition are known and shall be referred to herein as devices comprising an “onboard” composition. Reference can be made to WO 07/056,509 which teaches the inclusion of an onboard soap composition in a wet shaving razor. It is also known to provide a wet shaving razor incorporating an onboard skin-engaging composition comprising large quantities of hydrophilic polymers, such as polyethylene oxide, to lubricate the skin. Reference is made, by way of example, to WO 97/02116 and WO 97/02117.

The patent applications referred to above relate to the provision of various advantages, such as additional lathering and soap-related benefits, or improved lubrication in the case of polyethylene oxide. It would alternatively or additionally be advantageous to be able to provide a skin moisturizing benefit via an onboard chemistry, especially to male users who may be less motivated to use skin moisturizers than females. The provision of a moisturizing benefit from an onboard chemistry may, however, have a number of difficulties associated with it.

Skin moisturization may be achieved in several different ways, but one important formulational route to achieving skin moisturization is to include materials which bind water, such as polyols. However, such materials derive their water-binding abilities in part from their significant hydrophilicity, which may render them unsuited for use in any context involving water, such as is the case during wet shaving—they may be washed away during the initial stages of a shave. An alternative approach might be to use occlusive, hydrophobic materials which cover the skin and therefore act to retain water already present therein. These materials are emollients which are less likely to be washed away during use in a highly aqueous environment. WO 06/108522, discloses the use of small amounts of hydrophobic emollients in an onboard chemistry. However, formulations comprising significant proportions of hydrophobic emollients may give rise to increased drag across the skin, due to the affinity of such materials with the hydrophobic skin surface. Users report that increased drag tends to increase discomfort during shaving. US 2009/0223057 discloses shaving aid strip compositions comprising polyoxyethylene for similar reasons to the above-discussed prior art. In order to improve the longevity of the shaving aid, the polyoxyethylene is mixed with amphipathic, but generally hydrophobic materials, such as fatty alcohols.

### SUMMARY OF THE INVENTION

According to a first aspect of the invention, a razor cartridge is provided comprising a housing having a base which is connectable to a handle, two or more blades, whose tips are aligned in a cutting plane to cut in a cutting direction, and a moisturizing block disposed before and/or after the blades in the cutting direction, the moisturizing block having a skin-contacting surface, wherein: the moisturizing block comprises at least 50% lipophilic materials by weight of the moisturizing block from about 0.01% to 5% of an anti-irritation agent comprising a zinc pyrithione, a zinc salt, or a combination thereof.

According to a second aspect of the invention, a razor is provided comprising a cartridge according to the first aspect of the invention.

According to a third aspect of the invention, the use of the razor according to the second aspect of the invention is provided, to remove hair and moisturize the skin.

### BRIEF DESCRIPTION OF THE DRAWINGS

FIG. 1. is a view of a razor cartridge according to the invention.

### DETAILED DESCRIPTION OF THE INVENTION

According to the invention, a razor cartridge is provided comprising at least two blades whose tips are aligned in a cutting plane to cut in a cutting direction. The razor cartridge comprises a housing having a base. The base is connectable to a handle via means known to the man skilled in this art and which need not be elaborated here.

The razor cartridge according to the invention comprises a moisturizing block. The moisturizing block is configured to be erodable, such that it erodes during and as a result of shaving human skin to leave moisturizing deposits on the skin. As used herein, the word “erodable”, when used in relation to the moisturizing block, includes a moisturizing block having a Chatillon Hardness at 25° C. of 0.50-3.25 kg, preferably 0.75-3.00 kg, more preferably 1.00-2.50 kg, measured according to the protocol provided hereinbelow. Within these ranges, beneficial rates of wear may be achieved. In order to provide effective moisturization, the moisturizing block comprises at least 50%, preferably from 60% to 95% and more preferably from 70% to 90% lipophilic materials by weight of the moisturizing block.

The lipophilic materials may be liquid, semi-solid and/or solid at room temperature and may comprise one or more hydrocarbons, fatty acids, fatty alcohols, esters, triglycerides, fats, butters, waxes, lipophilic skin active agents or mixtures thereof.

Advantageously, if solids or semi-solids are present, then the moisturizing block comprises less than 20% and preferably less than 5% by weight of the moisturizing block of materials, and more preferably no materials at all, having a melting point of more than 100° C. This is because excessive quantities of such materials may render the moisturizing block inflexible and therefore liable to crack during manufacture and/or use.

Liquid, semi-solid, or solid hydrocarbon lipophilic materials which may be comprised within the moisturizing block include straight chain, branched chain, saturated and unsaturated hydrocarbons and mixtures thereof and they may comprise natural or synthetic hydrocarbon emollients and mixtures thereof. Preferred natural hydrocarbon emollients include petrolatum, mineral oil and mixtures thereof. Preferred synthetic hydrocarbon emollients include branched chain hydrocarbons, such as isohexadecane (such as Arlamol HD™ from Croda) and Polydecene (such as Puresyn 2™ from Exxon Mobil).

Liquid, semi-solid, or solid fatty alcohol or fatty acid emollients which may be comprised within the moisturizing block include saturated and unsaturated higher alcohols, especially C₁₂-C₃₀ fatty alcohols and fatty acids, especially lauric, myristic, palmitic, stearic, arachidic or behenic. Liquid, semi-solid, or solid ester emollients which may be comprised within the moisturizing block include esters of a C₁₂-C₃₀ alcohol and mixtures thereof, especially isopropyl myristate, isopropyl isostearate and mixtures thereof.

Liquid, semi-solid, or solid triglyceride emollients which may be comprised within the moisturizing block include synthetic or natural triglycerides, especially natural triglycerides derived from sunflower, avocado, olive, castor, coconut, cocoa and mixtures thereof. More preferred are coconut-derived triglycerides, such as the commercially available materials Myritol™ 312 and 318 (Cognis), Estasan™ (Croda) and Miglyol™ (Sasol).

Liquid, semi-solid, or solid fat and butter emollients which may be comprised within the moisturizing block include coconut butter, shea butter and mixtures thereof.

Liquid, semi-solid, or solid wax emollients which may be comprised within the moisturizing block include paraffin wax, microcrystalline wax, candellila, ozokerite and mixtures thereof, preferably paraffin wax. Advantageously, moisturizing block comprises some wax because waxes may bestow improved hardness and erodability to the moisturising block, although, as discussed above, the presence of too much wax may render the composition inflexible and therefore liable to crack during manufacture and/or use. Preferably, the moisturising block comprises from 2% to 20% and more preferably from 3% to 15% wax by weight of the moisturising block.

Liquid, semi-solid, or solid lipophilic skin active agents which may be comprised within the moisturizing block include oil soluble vitamins and agents which have activity on skin, such as vitamin E derivatives, including vitamin E acetate and tocopherol nicotinate; oil-soluble vitamin A derivatives, such as retinyl palmitate; lanolin; ceramides; sterols and sterol esters; salicylic acid; camphor; eucalyptol; essential oils and mixtures thereof.

### Anti-Irritation Agent

In one embodiment the shave preparation composition of the present invention further comprises an anti-irritation agent. The anti-irritation agent can be pyrithione or a polyvalent metal salt of pyrithione, or a mixture thereof. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm. Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

In one embodiment, the anti-irritation agent is a solid particle in the form of a platelet as described here.

The composition of the present invention optionally includes an effective amount of a zinc salt. Preferred embodiments of the present invention include an effective amount of a zinc salt having an aqueous solubility within the composition of less than about 25%, by weight, at 25° C., more preferably less than about 20%, more preferably less than about 15%. Preferred embodiments of the present invention include from 0.001% to 10% of a zinc salt, more preferably from 0.01% to 5%, more preferably still from 0.1% to 3%. In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

Those of skill in the art will understand that the anti-irritation agent of the present invention can also have other benefits which may be desirable from a skin care composition, including but not limited to malodor control and/or anti-bacterial benefits depending on whether the composition is left on skin or rinsed off.

Without intending to be bound by theory, it is believed that these anti-irritation agents can provide various benefits including reduction or control of irritation as well as certain malodor control. In one embodiment, the composition further comprises other agents such as malodor control agents. The malodor active of the present invention is capable of providing an antimicrobial benefit. Such malodor actives are capable of destroying microbes, preventing the development of microbes or preventing the pathogenic action of microbes. A safe and effective amount of a malodor active may be added to the intimate cleansing product, at from about 0.001% to about 10%, or from about 0.01% to about 5%, or from about 0.05% to about 2%, or from about 0.1% to about 1%, or from about 0.3% to about 0.7%, or about 0.5% by weight of the composition.

Examples of malodor actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, clotrimazole, and mixtures thereof.

The moisturizing block according to the invention may be disposed before and/or after the blades in the cutting direction, but is preferably disposed after the blades. The moisturizing block comprises a skin-contacting surface which may be planar or may define a shallow convex and/or concave curve. Advantageously, the skin-contacting surface is planar, because a planar surface may provide a higher surface area for deposition than a convex or concave surface. The skin-contacting surface slopes away from the blades towards the base of the razor cartridge. This feature may reduce the drag experienced by users, while facilitating deposition of moisturiser by the moisturising block. Advantageously, the skin-contacting surface slopes such that all tangents to the skin-contacting surface slope at an acute angle, α, to the cutting plane. More advantageously, the angle, α, is from 5° to 15°.

The moisturizing block may additionally comprise a structuring polymer. Preferably, the moisturizing block comprises from 2% to 50%, preferably from 3% to 40%, more preferably 4% to 12% of structuring polymer by weight of the moisturizing block.

As defined herein, the structuring polymer is not regarded as being one of the “lipophilic materials” as defined above and should be ignored for the purposes of calculating the percentage weights of the “lipophilic materials”.

Advantageously, the structuring polymer comprises a block copolymer. More advantageously, the block copolymer comprises a di-block copolymer, a tri-block copolymer, a multi-block copolymer, a radial block copolymer, a random block copolymer, or a mixture of these polymers.

In the case in which the block copolymer comprises a tri-block copolymer, then the tri-block copolymer preferably comprises a linear ABA tri-block polymer. Without wishing to be bound by theory, applicants believe that the A blocks aggregate creating domains, within which the moisturizing hydrophobic phase may accumulate, connected together by the B-blocks. This structure may provide an appropriate hardness to bestow the requisite wear properties to the moisturizing block, while also being flexible enough to be processed and not to crack or break during processing and/or use.

Advantageously, the linear ABA block copolymer comprises styrene-butadiene-styrene (SBS) block copolymer, styrene-isoprene-styrene (SIS) block copolymer, styrene-ethylenebutylene-styrene (S-EB-S) block copolymer, or mixtures thereof. More advantageously, the linear ABA block copolymer preferably comprises styrene-ethylenebutylene-styrene (S-EB-S) block copolymer. More advantageously still, the weight ratio of styrene to butadiene in the S-EB-S is in the range 20:80 to 40:60 and preferably around 30:70.

Particularly useful commercially available ABA block copolymers include Versagel™ materials available from Penreco and the Kraton™ G series, especially G-6150, G-1651, G-1652 and 1654.

As discussed above, the structuring polymer comprised within the moisturizing block may comprise a random block copolymer. An example of a suitable random block copolymer is ethylene vinyl acetate (EVA) which is a copolymer of ethylene and vinyl acetate. Advantageously, the amount of ethylene comprised within the EVA polymer is from 65-90%, preferably from 70-85% by weight of the EVA to give beneficial wear properties. A commercially available range of EVA is called Elvax™, which is commercialised by DuPont.

The moisturizing block may comprise one or more additional components which bestow a suitable melt viscosity to the composition, such as oil phase gellants, to facilitate improved processing, provided that the additional component(s) do not significantly reduce the hardness or erodability of the moisturizing block. Examples of such components are trihydroxystearin, which is commercially available as Thixcin R™ (manufactured by Elementis Specialities), ethylene vinyl acetate (EVA) and mixtures thereof.

A moisturizing block according to the invention may be manufactured by heating the lipophilic materials to a suitable temperature to melt them, typically approximately 130° C., after which the structuring polymer is added and mixed well until the structuring polymer has dissolved. The mixture is then cooled, typically to approximately 90° C., after which any additional ingredients may be added. In a final step, the mixture is poured into suitable containers or moulds and allowed to cool to room temperature.

Once the mixture has set to form a moisturizing block, it may be affixed to a razor cartridge in any appropriate fashion. One such approach is to mould the moisturizing block directly onto the cartridge. Another approach involves directly or indirectly adhering the moisturizing block to the cartridge by means of an adhesive composition. One method of indirect adherence involves casting the moisturizing block onto a sheet of an appropriate substrate, such as an acetate sheet, which sheet is then adhered to the cartridge, for example mechanically or via an adhesive.

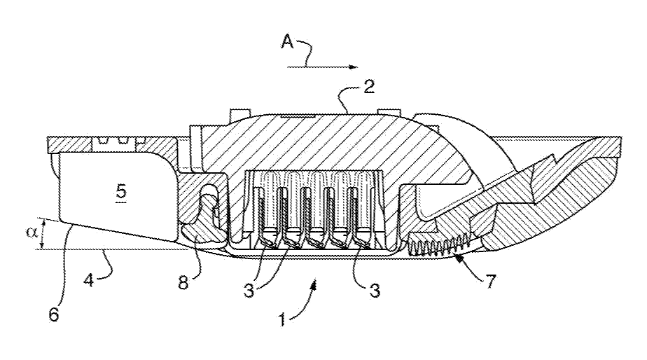

FIG. 1 depicts a non-limiting example of a razor cartridge (1) according to the invention. The cartridge (1) comprises a housing having a base (2) which is connectable to a handle using means known to the skilled person in this field. The cartridge (1) comprises blades (3) whose tips are aligned in a cutting plane (4) to cut in a cutting direction (A). The cartridge also comprises a moisturizing block (5). In this execution it is disposed after the blades (3), although it may instead or additionally be disposed before the blades (3). The moisturizing block (5) comprises skin-contacting surface (6), which slopes away from the blades (3) towards the base (2) at an angle, α, to the cutting plane (4). This embodiment also comprises a front skin-engaging element (7) and a rear skin-engaging element (8).

Chatillon Hardness test
Equipment: Chatillon TCD 200 equipped with a digital force gauge
Sample preparation
1. Fully melt and cast moisturizing block into 60 ml weigh boat (70 mm×70 mm×24 mm)
2. Store lipid at 25° C. overnight to equibrilate
3. Carefully remove moisturizing block from weigh boat prior to hardness testing

### A) Machine Preparation

1. Prepare Chatillon TCD 200 and digital force gauge according to manufacturers instructions.
2. Set the ramp speed to 47 mm/min
   B) Measuring the hardness value at 25° C.:
1. The pointed geometry should be attached to the shaft of ramp for this test method.
2. Place the moisturizing block as prepared above and on its side onto the metal base plate directly below the centre of the shaft of the ramp. The mid-point of the moisturizing block should be in line with the centre of the shaft of the ramp.
3. With the moisturizing block in place below the flat plate the speed set at 47 mm/min and the digital force gauge set at “C Peak” as above, depress the “Down” button on the Chatillon TCD200.
4. Stop the Chatillon TCD200 just as the probe touches the surface of the moisturizing block and set the distance counter to zero.
5. Reset the force gauge so that it reads zero
6. Depress the “Down” button on the Chatillon TCD200 until the distance counter reads 13 mm, record C Peak reading.

### Example

The following example discloses a moisturizing block according to the invention, which was incorporated into a razor cartridge as a strip disposed after the blade in the cutting direction. In use, when attached to a razor, the razor cartridge was observed to remove hair and moisturize the skin from which hair had been removed.

| Trade Name | INCI Name | % w/w |
|---|---|---|
| White soft paraffin | Petrolatum | 433.5 |
| Mineral oil | Paraffinum Liquidum | 433.5 |
| Kraton G1650E | Hydrogenated Styrene/Butadiene | 5.0 |
| | copolymer | |
| Thixcin R | Trihydroxystearin | 2.0 |
| Paraffin Wax SP206 | Paraffin | 5.0 |
| ZPT | 1-hydroxy-2-pyridinethione | 1.0 |

The composition of Example 1 was manufactured by heating the hydrocarbons and waxes to 130° C., then adding the linear ABA tri-block polymer (Kraton G1650E) and mixing well until polymer was fully dissolved. The mixture was then cooled to 90° C. and the Thixcin added, after which the mixture was moulded onto the cartridge and allowed to cool to room temperature.

The Chatillon Hardness of the formulation of Example 1 is 1.7 and the angle, α, was moulded to be 12.4°.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

What is claimed is:

1. A razor cartridge suitable for use on a razor handle, said razor cartridge comprising a housing having a base which is connectable to a handle, two or more blades, whose tips are aligned in a cutting plane to cut in a cutting direction (A), and a moisturizing block disposed before and/or after the blades in the cutting direction (A), the moisturizing block having a skin-contacting surface, wherein: the moisturizing block comprises at least 50% lipophilic materials by weight of the moisturizing block and from about 0.01% to 5% of an anti-irritation agent comprising a pyrithione, a polyvalent metal salt of pyrithione, and a mixture thereof.

2. The razor cartridge of claim 1, wherein said level of anti irritation agent is from about 0.25% to about 2.5%.

3. The razor cartridge of claim 1, wherein said anti-irritation agent comprises zinc pyrithione.

4. The razor cartridge of claim 3, further comprising from 0.001% to 10% of a zinc salt.

5. The razor cartridge of claim 3, wherein the zinc pyrithione is in platelet particle form having an average size of up to about 20 mm.

6. The razor cartridge of claim 3, wherein said zinc pyrithione is in the form of a platelet having a median particle diameter of about 0.5 microns to about 10, a mean particle diameter of about 0.5 to about 10 microns, and a thickness of about 0.6 to about 15 microns.

7. The skin engaging member of claim 6, wherein the mean particle diameter of the platelet ZPT is about 2 microns to about 4 microns.

8. The razor cartridge of claim 6, wherein the median particle diameter of the platelet ZPT is about 1 micron to about 5 microns.

9. The razor cartridge of claim 1, wherein the skin-contacting surface slopes away from the blades towards the base.

10. The razor cartridge of claim 1, wherein the moisturizing block comprises from 60% to 95%, preferably from 70% to 90% lipophilic materials by weight of the moisturizing block.

11. The razor cartridge of claim 1, wherein the moisturizing block comprises petrolatum, esters, triglycerides, waxes or mixtures thereof.

12. The razor cartridge of claim 1, wherein the moisturizing block comprises wax and preferably the moisturising block comprises from 2% to 20% and more preferably from 3% to 15% wax by weight of the moisturising block.

13. The razor cartridge of claim 1, wherein the moisturizing block has a Chatillon Hardness of 0.50 kg to 3.50 kg, preferably 0.75 kg to 3.00 kg, more preferably 1.00 kg to 2.50 kg.

14. The razor cartridge of claim 1, wherein the moisturizing block comprises from 2% to 50%, preferably from 3% to 40%, more preferably 4% to 12% of a structuring polymer by weight of the moisturizing block.

15. The razor cartridge of claim 14, wherein the structuring polymer comprises a block copolymer.

16. The razor cartridge of claim 1, wherein the moisturizing block is disposed after the cutting blades in the cutting direction (A).

17. The razor cartridge of claim 1, wherein said moisturizing block further comprises a malodor control agent.

18. A razor cartridge suitable for use on a razor handle, said razor cartridge comprising a housing having a base which is connectable to a handle, two or more blades, whose tips are aligned in a cutting plane to cut in a cutting direction (A), and a moisturizing block disposed before and/or after the blades in the cutting direction (A), the moisturizing block having a skin-contacting surface, wherein: the moisturizing block comprises at least 50% lipophilic materials by weight of the moisturizing block and from about 0.01% to 5% of an active comprising a solid particle in platelet form having an average size of up to about 20 μm.

19. The razor cartridge of claim 18, wherein said solid particle is in the form of a platelet having a median particle diameter of about 0.5 microns to about 10, a mean particle diameter of about 0.5 to about 10 microns, and a thickness of about 0.6 to about 15 microns.

20. The razor cartridge of claim 18, wherein the mean particle diameter of the platelet is about 2 microns to about 4 microns.
